(19) United States
(12) Patent Application Publication
(10) Pub. No.: US 2026/0199568 A1
(43) Pub. Date: Jul. 16, 2026

(54) Breast Pump

(71) Applicant: MEDELA HOLDING AG, Baar (CH)

(72) Inventors: Carsten Faltum, Vitznau (CH); David Y. Cho, Arlington Heights, IL (US)

(21) Appl. No.: 19/133,651

(22) PCT Filed: Dec. 5, 2022
(86) PCT No.: PCT/IB2022/000741

§ 371 Date: May 28, 2025

(51) Int. Cl. A61M 1/06

(52) U.S. Cl. CPC A61M 1/067; A61M 2202/0014; A61M 2202/04; A61M 2205/3331; A61M 2205/3592; A61M 2205/502; A61M 2205/8206; A61M 2209/088; A61M 2210/1007

(57) ABSTRACT

The invention generally relates to breast pump including a breast shield adapted to at least in part receive a breast of a lactating user, an aggregate for generating suction for expressing milk from the breast, a milk container providing a reservoir for the expressed milk and a controller for controlling the aggregate. For reducing weight to be borne by the bra and noise noticeable to the user the invention proposes an in-bra breast pump unit and an aggregate housing, which is in fluid communication with the in-bra breast pump unit but is not mechanically linked with the breast pump unit. The in-bra breast pump unit is adapted to at least in part fit inside the bra and includes the breast shield and the milk container. The aggregate is assigned to the aggregate housing.

The present invention relates to a breast pump and in particular to an in-bra breast pump which can be used to express milk from the breast of a lactating user without need to support the unit being in direct contact with the breast.

Such in-bra pump is also called a hands free pump as the mother is not forced to manually assist the breast pump during operation.

WO 2018/229 504 A1 proposes an in-bra breast pump having a housing receiving an aggregate, a control and a power source for energizing the aggregate and the control. A breast shield and a milk container are connected to the housing. In other words, all components of the breast pump are connectable to form an in-bra breast pump unit, which can be suspended by the bra of the user.

The prior art breast pump has the disadvantage of being fairly heavy and thus uncomfortable for the user. Moreover, the breast pump generates noise and vibration. In particular, the power source usually generates a certain amount of heat which is likewise discomfortable for the user of the breast pump.

The present invention wishes to provide a proposal to at least in part overcome the above disadvantages.

As a solution to this object, the present invention provides a breast pump including a breast shield adapted to at least in part receive a breast of a lactating user. As known in prior art, the breast pump has a milk container providing a reservoir for receiving expressed milk. The milk is expressed by suction generated by an aggregate. According to the inventive concept, the breast shield and the milk container are adapted to be joined to form an in-bra breast pump unit, which in-bra breast pump unit is adapted to at least in part fit inside a bra of the user. This means that the in-bra breast pump unit of the present invention is designed to be suspended and thus held by the bra of the user. The dimensions of the in-bra breast pump unit may be partially larger than the textile material forming the bra. In other words, the in-bra breast pump unit may project beyond the contour of the bra. Nevertheless, the in-bra breast pump unit is designed to receive at least a part of the breast of a lactating user, in particular the nipple of such breast, without need to support the in-bra breast pump unit other than with the bra.

According to the concept of the present invention, the aggregate of the breast pump is provided in an aggregate housing, which is in fluid communication with the breast pump unit but is not mechanically linked with such breast pump unit. In other words, the aggregate housing is a separate component of the inventive breast pump, which component does not need to be held by the bra of the user. The aggregate housing may be placed on any surface next to the user or may be adapted to be worn by the user, e.g. by connecting to a belt or the waistband of a trouser or a skirt.

The breast pump of the present invention provides a lower weight than the breast pump of prior art to be suspended by the bra. The aggregate as the major source of vibration and noise can be placed further remote from the ear of the user.

According to a preferred embodiment of the present invention, the in-bra breast pump unit comprises a housing receiving a power source for energizing the aggregate. This housing is detachably coupled with said breast shield and said milk container. Coupling may be provided directly or indirectly. With a direct coupling, the housing is in direct mechanical contact with the breast shield or the milk container. An indirectly coupled housing is mechanically linked with the breast shield and the milk container without necessarily having a direct mechanical link which attaches the housing to the breast shield and/or the milk container. The housing may be received within a receptacle provided within the breast shield and/or the milk container. The housing may be arranged between the breast shield and the milk container. Most preferably, the housing will be coupled such that the housing is provided within an outer surface of the breast pump projecting the body of the user, which outer surface imitates the shape of a female breast. In other words, the in-bra breast pump unit shall preferably be designed as known in prior art with a generally conical contact surface for the breast provided by the breast shield, which breast shield has a nipple tunnel for receiving the nipple, which breast shield at least in part is received within or projects into the milk container. The housing may be arranged above or below the milk container, which orientation refers to the general use condition of the breast pump in which the nipple tunnel extends essentially horizontally, i.e. the user is assumed to have a position in which at least the torso is upright, i.e. vertically extended (standing or sitting). The outer surfaces projecting from the body of the user shall be provided without step to generate a smooth outer surface projecting from the body of the user. The outer surfaces of the in-bra breast pump unit other than the conical contact surface may be conical or flat and conical.

The controller may be provided within the housing or the aggregate housing. The later provides the further advantage that heat generated by the controller during control of the aggregate will not reach the body of the user, in particular the breast. The breast of a lactating user is in particular sensible.

The controller may be split between the housing and the aggregate housing. In other words, some portions of the controller controlling the aggregate may be provided in the aggregate housing while those portions of the controller assigned to in particular a user interface may be provided within the housing. Such constitution will allow the user to easily find and access the user interface when using the breast pump. The user interface may be provided with a light source to illuminate buttons of the user interface. The housing may as well have optical indicators allowing the user to obtain information on the operational status of the breast pump. Respective information may include fill level information giving information on the volume of expressed milk received within the reservoir. The illumination means mentioned above will allow the user to observe such information in the dark and/or allow swift and secure identification of different elements or functions of the user interface.

According to a preferred embodiment of the present invention, a suction line is coupled to said aggregate housing and said in-bra breast pump unit. In other words, such suction line extends between the aggregate housing and the in-bra breast pump unit. Respective suction line may preferably be coupled to the housing or the milk container. Alternatively or additionally, a power line may be coupled to said aggregate housing and said in-bra breast pump unit. Such power line will allow transmittal of electric energy between the power source and the aggregate and/or the control. Further, physical lines may be provided between the aggregate housing and the in-bra breast pump unit, e.g. to exchange data between those two separate items of the breast pump.

Data and/or energy may likewise be exchanged between the aggregate housing and the in-bra breast pump unit by wireless means. A wireless energy connection may be provided via power transfer modulated by control signals.

In order to reduce the number of overall strings extending between the in-bra breast pump unit and the aggregate housing, the breast pump preferably has a unitary connection line which integrates the suction line and the at least one power line. Preferably, the unitary connection line comprises any line for exchanging data, fluid or energy between the in-bra breast pump unit and the aggregate housing. The unitary connection line will be the only physical connection between the two components of the breast pump.

According to the present invention, the power source mentioned above for energizing the aggregate may as well be received within the aggregate housing. This embodiment is suitable for wireless transmittal of power to the housing as the controller and/or the user interface may be the only consumer of electric energy of the in-bra breast pump unit.

Further details and advantages of the present invention will become apparent from the following description of an embodiment in connection with the drawing. In the drawings:

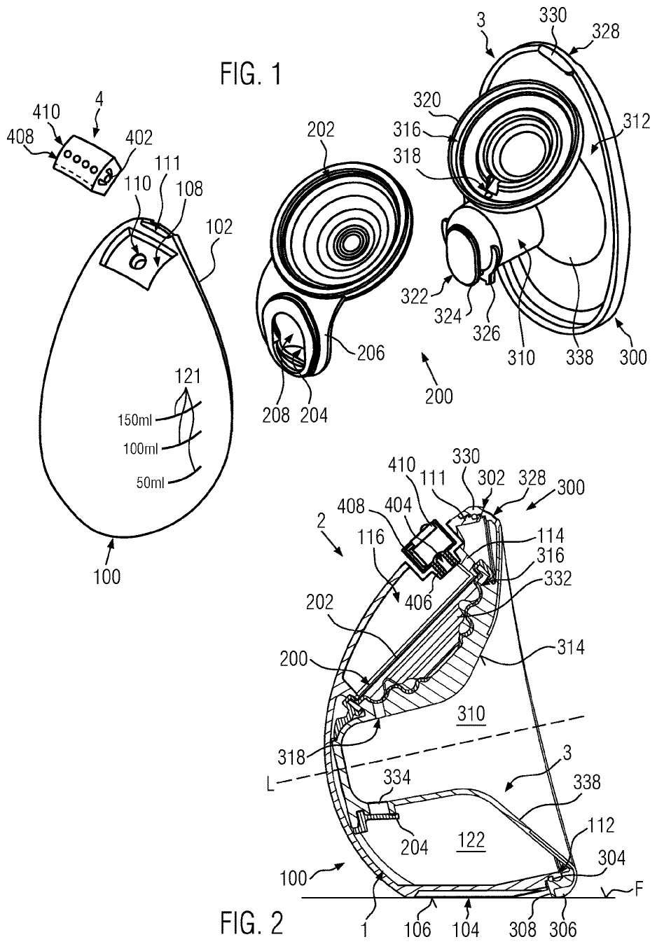

FIG. 1 is a perspective exploded view of the embodiment;

FIG. 2 is a cross-sectional view of the embodiment of FIG. 1, which cross-sectional view contains the longitudinal axis of the nipple tunnel and extends parallel to a plane comprising the vertical axis;

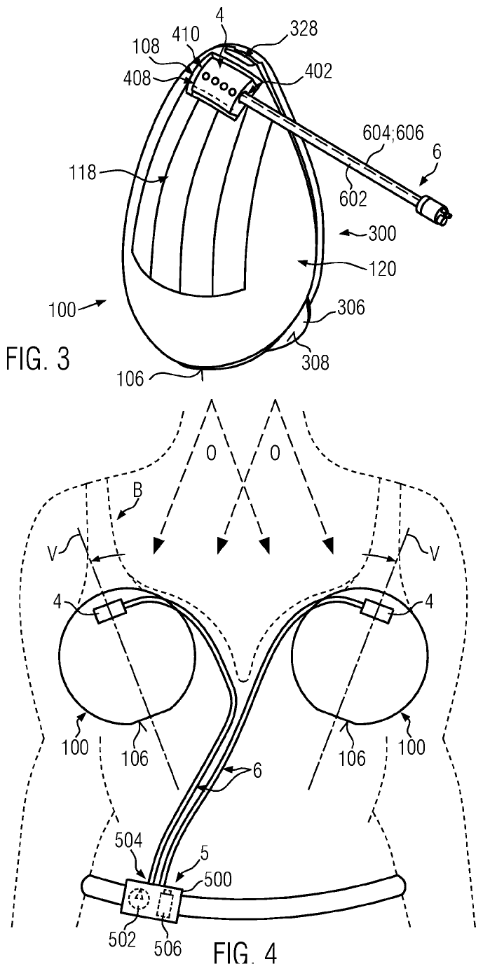

FIG. 3 is a perspective view of the front side of the embodiment of FIGS. 1 and 2 and

FIG. 4 is a front view showing two different use situations of the embodiment of FIGS. 1 and 2.

In the Figures, reference numeral 100 identifies an outer shell element 100, reference numeral 200 identifies a media separation element and reference numeral 300 identifies a breast shield element providing a breast shield 3. The breast shield element 300 provides a lit cooperating with the shell element 100 to define a milk container 1 of an in-bra breast pump unit 2. Reference numeral 4 identifies a housing of the in-bra breast pump unit 3, which housing 4 is in fluid communication and data communication with an aggregate unit 5 shown in FIG. 4 via a connection line 6. The aggregate unit 5 is separate from the in-bra breast pump unit 3 and not mechanically linked with said in-bra breast pump unit 3. The housing 4 is detachable from the milk container 1.

The outer shell element 100 is spherical and defines a plane interface 102 surrounded by a circumferential rim 302 of the breast shield element 300. A sealing arrangement in form of a circumferential seal 304 is provided between the interface 102 and the circumferential rim 302. At a bottom of the in-bra breast pump unit 2, the breast shield element 300 has a wing 306 defining a detachment surface 308. Adjacent to said detachment surface 308, the outer shell element 100 provides a flat recess 104 as an interruption to the spherical outer surface of the outer shell element 100, which recess 104 provides a standing surface 106. For opening the embodiment as depicted in FIG. 2, the user will grasp around the outer shell element 100 such that the thumb of the hand grasping around the outer shell element 100 will lay against the holding surface 106. The thumb of the other hand will cooperate with the detachment surface 308 while the fingers of the hand will at least in part cooperate with the circumferential rim 302 at the top of the pump to remove the breast shield element 300 from the outer shell element 100.

Near the top, the outer shell element 100 provides a receptacle 108 formed as a recess within the spherical outer surface and adapted to receive the housing 4. The housing 4 i.e. a circumference thereof is provided with a tube interface 402, which can be connected with the unitary connection line 6 depicted in FIG. 3. The receptacle 108 has a longish extension. Due to the spherical constitution of the outer shell element 100, the opposing lateral rims surrounding the receptacle 108 are arranged lower than the axial extension of the tube interface 402. Thus, the unitary connection line 6 can extend form the housing 4 in a lateral extension as shown in FIG. 3. The housing 4 has a central bore 404 extended by a protrusion 406 sealingly cooperating with a working medium inlet 110 provided in the center of the receptacle 108 (compare FIG. 2). Thus, the housing 4 can be arranged in an alignment 180° tilted around the working medium inlet to provide connection of the connection line 6 in a lateral extension opposite to the extension shown in FIG. 3.

The breast shield element 300 of the present embodiment is a co-injection molded unitary element having a nipple tunnel 310 made of a transparent material and a flange section 312 made of an opaque material, which flange section 312 provides a contact surface 314 to at least in part contact the breast of the user. Due to the transparent properties of the nipple tunnel 310, the user can confirm proper alignment of the nipple within the nipple tunnel 310. Above the nipple tunnel 310, the breast shield element 300 defines a pumping chamber housing 316 which via a pumping inlet 318 is in fluid communication with the nipple tunnel 310. The pumping chamber 316 has a flat upper rim 320. The upper rim 320 is ring-shaped.

An inner end 322 of the nipple tunnel 310 is provided with a securing interface 324. The bottom side of the nipple tunnel 310 is projected by a valve receptacle 326.

At the top of the breast shield element 300, there is provided an open spout 328, which is projected by a snapping hook 330 provided by the breast shield element 300 and cooperating with a snapping counter surface 111 provided by a ring segment 112 of the outer shell element 100 with the circumferential seal interdisposed.

The media separation element 200 has a membrane 202 and a valve 204 being connected by a connection section 206 having a securing opening 208. The membrane 202, the valve 204 and the connection section 206 are realized in a single unitary element i.e. the media separation element 200.

As visible from FIG. 2, the outer shell element 100 has a circumferential rim 114 projecting inwardly from the concave inner surface of the outer shell element 100.

For assembly, the media separation element 200 will be secured to the inner end 322 of the nipple tunnel 310 by inserting the securing interface 324 at least in part into the securing opening 208. Mating connection surfaces of the media separation element 200 and the securing interface 324 will provide attachment of the two elements 100, 200. Thereafter, the membrane 202 will be tilted and secured against the upper rim 320 to close a pumping chamber 332. As a result and as visible from FIG. 2, the membrane 202 grasps around the upper rim 320 for proper attachment of the membrane 202 against the breast shield element 300, specifically the pumping chamber housing 316. On the other side, the valve 204 will be tilted to be connected within the valve receptacle 326 and abut against a milk outlet 334 provided on an outer circumference of the nipple tunnel 310.

In the further course of the assembly, the preassembled group comprising the media separation element 200 and the breast shield element 300 is inserted into the outer shell element 100. In the course of this assembly, the circumferential rim 114 will abut the membrane 202 so that the membrane 202 is sealed between the breast shield element 300 and the outer shell element 100.

Thus, a working fluid chamber 116 is provided within the outer shell element 100, which working fluid chamber 116 is in communication with the tube interface 402 via the working medium inlet 110.

FIG. 3 elucidates formation of the outer shell element 100 of two plastic materials with different transparent properties. For the following discussion, referral is made to a longitudinal axis of the nipple tunnel identified with reference numeral L in FIGS. 2 and 3 and a vertical axis V extending perpendicular to the longitudinal axis L and extending in the same plane, which vertical axis defines the sectional view of FIG. 2. The embodiment is symmetrical with respect to the vertical axis V.

The same applies to the different sections of the outer shell element 100. A first section 118 covers the nipple tunnel 310 and, thus, is arranged in a central portion of the outer shell element 100. The first section 118 is made of a frosted transparent thermoplastic material. A second section 120 is provided laterally to the first section 118 and is made of slightly frosted transparent plastic material having a higher degree of transparency than the first section 118. The two sections 118, 120 are joined by co-injection molding. The lateral sides of the second section 120 are each provided with scale-lines 121 identifying the amount of milk collected within the pump. Alternatively scale-lines may be provided on the breast shield 4. In the present embodiment, the second section 120 is U-shaped and also extends over a central area of the outer shell element 100. In the present embodiment, the second section 120 provides the bottom of the outer shell element 100.

FIG. 1 exemplifies the breast shield element 300 being made of two different materials. The nipple tunnel 310 as well as a conical section 338 emerging from the nipple tunnel 310 are made of a clear transparent material, while the flange section 312 is made of a frosted or opaque plastic material. This constitution allows the user to check nipple alignment within the nipple tunnel 310 through the breast shield element 300.

In use, which is shown in FIG. 4, the breast pump is arranged with the vertical axis V slightly inclined outwardly below the breast pump. Thus, a view O of the user will be directed to the lateral sides of the outer shell element and, thus, the second section which will allow the user to observe milk flow and nipple alignment as well as e.g. valve operation. FIG. 4 also shows that the connection line 6 for left and right breast use of the in-bra breast pump unit 2 is directed towards the center of the body to be connected to the aggregate unit 5 thanks to the two different orientations in which the housing 4 is rotated by 180° within the receptacle 108. For simultaneous operation, the aggregate 502 may be adapted to be connected or connectable to two connection lines 6, each leading to a separate housing 4 one being assigned to the right breast, the other being assigned to the left breast.

The outer shell 100 is avocado-shaped. The outer shell element 100 has the largest width approximately at level of the longitudinal axis L, the width being an extension perpendicular to the plane containing the vertical axis and the longitudinal axis. The outer shell element 100 and thus the breast shield element have the largest extension in height direction in the plane containing the vertical axis V. The longitudinal axis L is provided below the middle height of the outer shell element 100, the height being the extension parallel to the vertical axis. Due to this, the center of gravity of the pump is essentially leveled with the longitudinal axis or slightly below the longitudinal axis L. Due to this center of gravity, the pump is inclined to assume a positon in which the open spout 328 assumes the highest point of the pump, whereas the holding surface 106 assumes the lowest point. In fact and as evident from FIG. 2, this standing surface 106 securely supports an upright position of a pump being placed on a flat horizontal surface F.

Due to the avocado shape of the outer shell element 100, the breast pump of the embodiment can be stored at least in part within a bra B-FIG. 4. Thus, the in-bra breast pump unit 2 is a hands-free pump.

In operation, the negative pressure of the aggregate 502 cyclically evacuates the working fluid chamber 116, which causes the membrane 202 to expand either towards the working fluid chamber 116 or towards the pumping chamber 332. The aggregate 502 may comprise an electric motor, which e.g. can be driven by a Piezo drive or a magnetic drive. For generating a negative pressure, the aggregate 502 may comprise or be connected to a membrane or a piston. In general, the source of the negative pressure is located within the aggregate housing 500. Through the pumping inlet 318, the pressure profile thus generated in the pumping chamber 332 is transferred into the nipple tunnel 310. Due to this, milk is expressed, which milk is lead down into the reservoir 122 sandwiched between the outer shell element 100 and the breast shield element 300. The reservoir 122 in fact assumes the entire inner space within the outer shell element 100 which is not covered by material of the breast shield element 300 or the media separation element 200 or the volume of the working fluid chamber 116 and the pumping chamber 332. The reservoir 122 is in communication with the atmosphere through the open spout 328. As the open spout 328 is arranged at the top of the in-bra breast pump unit 2 and as the open spout 328 has a rather small dimension, spillage of milk expressed and contained within the reservoir 122 can be avoided.

The housing 4 contains a controller 408 in form of a PCB and in data communication with elements of a user interface 410, which elements are provided on an outer front surface of the housing 4 directed to the user of the in-bra breast pump unit 2 in use. The connection line 6 contains a suction line 602 in form of tube, a data line 604 providing a data connection between the controller 408 and the aggregate 502 and a power line 606 for supplying the controller 408 with electric energy of a power source 506 in form a battery, which is arranged in the aggregate housing 500. Alternatively, the power line may connect the aggregate 502 with a power source (not shown) within the housing 4. Evidently, the aggregate unit 5 is provided off the PCB of the controller 408. The data line 604 and the power line 606 may be provided by one physical entity. The user interface 410 may comprise at least one optical indicator for indicating an operational condition e.g. of the aggregate 502 or the fill level of the reservoir 122. For this, the housing 4 may be equipped with a fill sensor or at least an interface of such fill sensor. Such fill sensor may be connected with the outer shell element 100, preferably formed integrally with said outer shell element 100 to allow dishwashing of the outer shell element 100. The user interface 410 may comprise at least one element 410 like a button to change operational conditions of the aggregate 502.

After expressing milk, the pump will be taken out of the bra B and can rest on the standing surface 106 in an upright position. The expressed milk can be poured out through the open spout 328 by rotating the pump upside down. During pumping operation and filling up the reservoir 122 with milk, air can vent through the open spout 328.

In the present embodiment, the outer surface of the in-bra breast pump unit 2 is provided essentially by the outer shell element 100 and the breast shield element 300 only. The breast shield element 300 provides the breast shield 3 adapted to be laid against the beast of the user, specifically by means of the contact surface 314.

The outer shell element 100 and the breast shield element 300 are connected with each other. The outer periphery of breast shield element 300 is level with the outer periphery of the outer shell element 100. Free outer edges formed by the outer periphery of the breast shield element 300 and/or the outer shell element 100 are avoided in favor of a smooth outer housing 2 of the breast pump. The outer periphery of the outer shell element 100 is grasped around by and received within the outer periphery of breast shield element 300.

The working fluid chamber 116, the pumping chamber 332, and the milk compartment 122 are exclusively defined by the three individual but unitary elements discussed in detail above, i.e. the outer shell element 100, the media separation element 200, and the breast shield element 300. As the media separation element 200 as a single unitary element provides the membrane 202 as well as the valve 204, the breast pump of the present invention can be manufactured with a reduced number of parts. The media separation element 200 can easily be mounted on the breast shield element 300. Assembly of the in-bra breast pump unit 2 is facilitated due to the reduced number of individual parts to be handled as well as the possibility to temporarily attach the media separation element 200 against the inner end 322 of the nipple tunnel 310. Due to the cooperating surfaces provided by the circumferential ring 114 and the upper rim 320, the membrane 202 is securely sandwiched to seal the fluid working chamber 116 and the pumping chamber 332 after assembly of the pump.

Transparent or slightly frosted sections of the breast pump unit 2 allow visibility through the outer shell element 100 and at least portions of the breast shield element 300, in particular the nipple tunnel 310 to observe nipple alignment and/or milk flow and seize thereof indication termination of milk flow.

Evidently, the aggregate 502 can be arranged physically separate of the in-bra breast pump unit 2 thereby relieving the user to also suspend the aggregate by the bra B of the user. The in-bra breast pump unit 2 is shaped to at least fit in part inside the bra of the user. In other words, the in-bra breast pump unit 2 can be arranged within the bra B and suspended by the bra B with the breast at least being received in part within the breast shield 3. The in-bra breast pump unit is a hands free unit. The aggregate unit 5 of the embodiment of FIG. 4 is suspended by a belt of the user.

### LIST OF REFERENCE SIGNS

1 milk container
2 in-bra breast pump unit
3 breast shield
4 housing
5 aggregate unit
6 connection line
100 outer shell element
102 interface
104 recess
106 standing surface
108 receptacle
110 working medium inlet
111 snapping counter surface
112 ring segment
114 circumferential ring
116 working fluid chamber
118 first section
120 second section
121 scale line
122 reservoir
200 media separation element
202 membrane
204 valve
206 connection section
208 securing opening
300 breast shield element
302 circumferential rim
304 circumferential seal
306 wing
308 detachment surface
310 nipple tunnel
312 flange section
314 contact surface
316 pumping chamber housing
318 pumping inlet
320 upper rim
322 inner end
324 securing interface
326 valve receptacle
328 open spout
330 snapping hook
332 pumping chamber
334 milk outlet
338 conical section
402 tube interface
404 bore
406 protrusion
408 controller
410 user interface
500 aggregate housing
504 aggregate interface
506 power source
602 suction line
604 data line
606 power line
L longitudinal axis of the nipple tunnel
V vertical axis
F flat surface
B bra
O view

1. A breast pump including a breast shield adapted to at least in part receive a breast of a lactating user, an aggregate for generating suction for expressing milk from the breast, a milk container providing a reservoir for the expressed milk, a controller for controlling the aggregate and a power source, characterized in that the breast shield and the milk container are adapted to be joined to form an in-bra breast pump unit adapted to at least in part fit inside a bra (B), the aggregate is provided in an aggregate housing, which is fluid communication with the in-bra breast pump unit but is not mechanically linked with said breast pump unit, wherein the controller and/or the power source are mechanically linked to the breast pump unit.

2. The breast pump according to claim 1, the in-bra breast pump unit comprising a housing receiving the power source for energizing the aggregate, which housing is detachably coupled with said breast shield and said milk container.

3. The breast pump according to claim 2, and said controller is provided within said housing.

4. The breast pump according to claim 2, and said controller is provided within said aggregate housing.

5. The breast pump according to claim 3, and said housing is provided with a user interface.

6. The breast pump according to claim 2, further comprising a suction line coupled to said aggregate housing and said in-bra breast pump unit.

7. The breast pump according to claim 6, further comprising a connection line integrating said suction line and said power line.

8. The breast pump according to claim 6, wherein one of the connection line, the suction line, or the power line is coupled to the aggregate housing and the in-bra breast pump unit.

9. The breast pump according to claim 1, wherein a power source for energizing the aggregate, which power source is received within the aggregate housing.

10. The breast pump according to claim 9, further comprising a control power source for energizing said controller, which control power source is received in the housing.

11. The breast pump according to claim 1, further comprising a wireless data and/or energy connection between the in-bra breast pump unit and the aggregate housing.

12. The breast pump according to claim 2, further comprising a power line coupled to the aggregate housing and the in-bra breast pump unit.
